(19) United States
(12) Patent Application Publication
(10) Pub. No.: US 2024/0287434 A1
(43) Pub. Date: Aug. 29, 2024

(54) INCUBATOR WITH SHAKING MECHANISM

(71) Applicant: LICONIC AG, Mauren (LI)

(72) Inventor: Cosmas MALIN, Mauren (LI)

(73) Assignee: LICONIC AG, Mauren (LI)

(21) Appl. No.: 18/573,526

(22) PCT Filed: Jun. 21, 2022
(86) PCT No.: PCT/EP2022/066909

§ 371 Date: Dec. 22, 2023

(30) Foreign Application Priority Data
Jun. 23, 2021 (CH) 0727/21

(51) Int. Cl. C12M 1/00; C12M 1/36; C12M 3/00; C12M 3/06

(52) U.S. Cl. CPC C12M 41/14; C12M 23/48; C12M 27/16; C12M 41/48

(57) ABSTRACT

An incubator for sample containers comprises a climate-controlled chamber with several storage racks located therein. Shaker units are provided for shaking the storage racks, each of them carrying several storage racks. A robot unit accesses the storage locations in the storage racks as well as an automated access door at the back of the chamber. A user door is located at the front of chamber. The shaker units and the storage racks are arranged on opposite sides of a vertical plane extending perpendicularly to the doors.

### TECHNICAL FIELD

The present invention relates to an incubator for samples in sample containers that comprises a temperature-controlled chamber with several storage racks arranged therein and a shaker mechanism for shaking the storage racks. The invention also relates to a method of operating such an incubator.

### BACKGROUND ART

EP1721964 describes an incubator of this type. Its shaker mechanism comprises a shaker drive shaking a support member. The support member carries several storage racks. Alternatively, individual support members for the storage racks may be provided.

### DISCLOSURE OF THE INVENTION

The problem to be solved by the present invention is to provide an incubator of this type that scales well with a larger number of storage racks as well as a method for operating the same.

This problem is solved by the incubator and method of the independent claims.

Accordingly, the incubator comprises at least the following elements:
A chamber: This is the chamber receiving the samples to be incubated.
A climate control unit: This unit is connected to the chamber to maintain a defined temperature in the chamber. Advantageously, it is also adapted to maintain an elevated humidity in the chamber.
Several storage racks arranged in the chamber: Each storage rack comprises several storage locations arranged on top of each other. The storage locations may be adapted to removably receive the sample containers, such as microtiter plates, tube racks, or flasks.
Shaker units: Each such shaker unit comprises a shaker drive and a support member connected to the shaker drive. The support member carries at least two of the storage racks.

Hence, in contrast to the prior art, there are several shaker units with each shaker unit carrying several storage racks. This allows to increase the number of storage racks while keeping the number of shaker units low. On the other hand, it is still possible to control the shaking individually for subsets of the storage racks. For example, when a sample container needs to be loaded into or unloaded from a storage rack, a single shaker unit may be stopped while the others can continue the shaking operation. Also, if desired, different shaking parameters can be applied to different subsets of the storage racks.

Advantageously, the incubator further comprises:
A user door: The user door provides access—for the user of the incubator—to the chamber. It may e.g. be used for maintenance work, such as for cleaning, and/or for removing or inserting storage racks. Advantageously, the height of the user door is therefore at least equal to the height of a storage rack.
A robot unit arranged in the chamber: This robot unit may be adapted to automatically access sample containers arranged in the storage locations. It comprises at least the following elements:
a) A vertical guide rail.
b) A carriage vertically displaceable along the guide rail.
c) An elevator drive connected to the carriage: It may be adapted to vertically displace the carriage in order to access a storage location at a given height.
d) A container access unit movably arranged on the carriage: This access unit may be designed to access individual locations at the height of the carriage.
e) An access drive connected to the access unit: This drive may be adapted to operate the access unit for accessing the containers.

In this case, a vertical plane extending perpendicularly to the user door (in its closed position), through the vertical guide rail, and through the carriage divides the chamber into first and a second section. A first subset of the storage racks is arranged in the first section, and a second subset of the storage racks is arranged in the second section. This allows the user to easily access the storage racks through the user door because they are either located to the left or to the right of the vertical guide rail and the carriage.

The incubator may further comprise an automated access door, which may be adapted for an automated transfer of sample containers between the chamber and an external system. In this case, the vertical plane advantageously intersects the access door, and the chamber is arranged between the access door and a user door. The robot unit may be adapted to move sample containers between the storage locations in the first and second section of the storage chamber and the access door, with the latter advantageously being located between the first and section sections.

The first subset of the storage racks is advantageously mounted to a first subset of the shaker units, and the second subset of the storage racks is advantageously mounted to a second subset of the shaker units, with the first and second subset not having any common shaker units. Hence, separate subsets of shaker units are provided for the two sections, which may e.g. allow the user to easily access individual shaker units through either the left or the right section of the chamber.

In an embodiment of the invention, at any given vertical level, there is no more than one shaker unit in the first section and no more than one shaker unit in the second section. Hence, each section has, for a given level, only one shaker unit in the first section and only one shaker unit in the second section.

The incubator may comprise several shaker units and storage racks at different vertical levels. This allows to use shorter storage racks that are easier to shake and/or to provide a larger number of individually controllable shaking zones.

The invention also relates to a method for operating such an incubator comprising the step of operating at least some of the shaker units at different speeds. In this context, “operating at different speeds” also includes the case where at least one of the speeds is zero, i.e. where at least one of the shaker units is switched off, while at least another one is shaking. It also includes the case where two shaker units are operated at different frequencies and/or amplitudes and/or differently shaped motion profiles. This allows to adapt the shaking in different parts of the incubator to different requirements. The operation with different speeds may be continuous or only extend over a certain period of time, such as e.g. over at least 10 seconds.

In particular, the invention may comprise at least the following steps:
Stopping the shaking of a first one of the shaker units while continuing the shaking of at least a second one of the shaker units. Then
Accessing one of the storage locations of the storage racks mounted on the first (non-shaking) shaker unit with a robot unit. This allows to access the storage location while the respective storage rack is at rest. At the same time, the other shaker unit(s) do not need to interrupt their shaking motion. “Access the storage location” may e.g. comprise depositing a sample container in the storage location or retrieving a sample container therefrom. Then
Resuming the shaking of the first one of the shaker units after ending the access. In other once, once the robot unit has finished access, the shaking motion of the first shaker unit is restarted.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1 shows a schematic front view of an incubator with its user door not shown,

FIG. 2 shows a sectional view along line II-II of FIG. 1,

FIG. 3 shows a view, from above, of a more detailed embodiment of an incubator, and

FIG. 4 shows yet another embodiment of an incubator.

### MODES FOR CARRYING OUT THE INVENTION

### Definitions

Terms such as “horizontal” and “vertical” as well as “above”, “below”, etc. refer to the intended orientation of the device where e.g. a foot section of the device is arranged at the bottom of the device and where the guide rail extends in vertical direction.

The term “shaking” refers to a repetitive, usually cyclic motion. Advantageously, it is limited to an amplitude (throw) of less than 5 cm, in particular of less than 1 cm. The amplitude is advantageously at least 0.1 mm. The fundamental frequency of the shaking is advantageously between 10 and 2000 rpm (i.e. cycles per minute), in particular between 20 and 1200 rpm. The motion may be harmonic or inharmonic. It may be in at least one horizontal and/or the vertical direction.

### First Embodiment

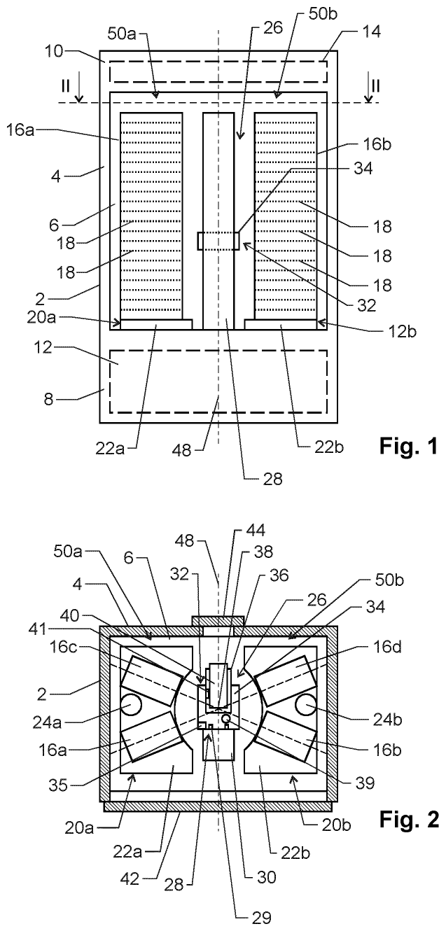

The embodiment of an incubator shown in FIGS. 1 and 2 comprises a housing 2 having insulated walls 4 enclosing a chamber 6. It further has a foot section 8 at the bottom of the device and a head section 10 at the top of the device.

The device further comprises a climate control unit 12, which may e.g. be arranged, at least in part, in foot section 8. Climate control unit 12 may e.g. comprise a resistive heater and/or a heat pump for keeping chamber 6 at a desired temperature. It may also comprise as a ventilation system. In addition or alternatively thereto, it may comprise a humidifier for controlling the relative humidity of the atmosphere in chamber 6.

For many incubation applications, climate control unit 12 maintains chamber 6 at a temperature between 0° and 50° C., such as between 20° and 40° C. yet other temperatures may be used as well.

In addition or alternatively thereto, climate control 12 may maintain a desired humidity in chamber 6, which is typically higher than the humidity of the environment, in order to reduce the increased tendency of evaporation of the samples due to the shaking. Advantageously, climate control 12 is adapted to maintain a relative humidity of at least 60%, in particular at least 90%, in particular at least 95% in chamber 6.

Accordingly, the invention also relates to a method comprising the step of maintaining a relative humidity of at least 60%, in particular at least 90%, in particular at least 95% in chamber 6 while operating the shaker units 20a, 20b.

The incubator also comprises a control unit 14, which may e.g. comprise a microcontroller and programming in a memory, adapted to electronically control the components of the incubator. In the shown example, it is—by way of example—arranged in head section 10.

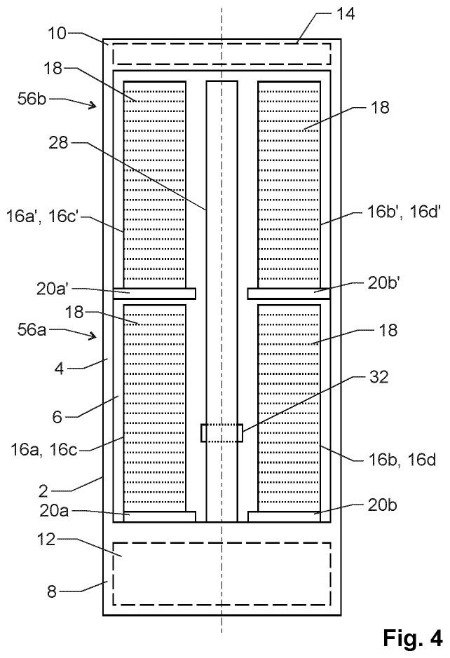

Several storage racks 16a-16d are arranged in chamber 6. Each storage rack is e.g. designed as shown in FIG. 4 of EP1721964, with this figure and the corresponding description of the storage racks and the shaker unit (see below) being incorporated herein by reference. It forms a plurality of storage locations 18 arranged above each other. (Only some of these storage locations are provided with reference numbers in FIG. 1.)

The present incubator further comprises two shaker units 20a, 20b, with two of the storage racks 16a-16d mounted to each of them.

The shaker units 20a. 20b may be designed as shown in FIG. 4 of EP1721964, which is incorporated by reference herein. They comprise a support member 22a, 22b, to which the storage racks 16a-16d are mounted, as well as a shaker drive 24a, 24b.

Each shaker drive 24a, 24b is adapted to shake its associated support member 22a, 22b, thereby shaking the storage racks 16a-16d mounted thereto.

In the shown embodiment, the support members 22a, 22b comprise a plate mounted to suitable horizontal guides and e.g. connected to a suitable eccentric drive shaft of shaker drive 24a, 24b. Reference is again made to EP1721964 for more detailed explanations.

Further, the incubator comprises a robot unit 26, which is adapted and structured to access the storage locations 18 and to load and unload them.

Robot unit 26 comprises a vertical guide rail 28, which e.g. comprises one or more tracks 29 and a support tower 30 mounted to the bottom of chamber 6. It further comprises a carriage 32 guided by and displaceable along guide rail 28.

In the present embodiment, carriage 32 comprises a base member 34 directly mounted to guide rail 28 and an elevator drive 35 adapted to vertically move carriage 32 along guide rail 28. Carriage 32 further comprises a table 36 rotatable in respect to base member 34 about a vertical rotation axis 38, and a shovel member 40 extendible, in respect to table 36, in a horizontal direction and radially to rotation axis 38.

The storage racks 16a-16d are facing rotation axis 38 with their storage locations 18 accessible by shovel member 40, e.g. as described in EP1721964.

Shovel member 40 forms a container access unit to access the sample containers in the storage locations. It may, as mentioned, be formed by a shovel to support and scoop up the sample containers from below, or it may e.g. be designed as a gripper with fingers to grip opposite sides of the sample containers. It may also comprise other means to engage the containers, such as hooks, electromagnets, or suction members.

Robot unit 26 further comprises a rotational drive 39 for rotating table 36 in respect to base member 34 about rotational axis 38 as well as an access drive 41 to extend shovel member 40 in respect to table 36.

The incubator further comprises a user door 42 at a front side thereof and an automated access door 44 at a back side thereof. Chamber 6 is located between user door 42 and access door 44.

Robot unit 26 is adapted to move sample containers between the storage locations 28 and access door 44, e.g. for exchanging them with an external container handling system.

Automated access door 44 is large enough to pass sample containers, but not much larger in order to reduce the impact of its opening on the climate within chamber 6. Advantageously, it covers an opening no larger than 10% of the back side of chamber 6.

User door 42 is, on the other hand, typically much larger, e.g. covering an opening having an area of at least 80% of the front side of chamber 6. It is typically used for manual maintenance of the components in chamber 6, such as for removing and inserting storage racks 16a-16d and/or shaker units 20a, 20b or for cleaning chamber 6.

As mentioned above, since tower 30 and guide rail 28 are located in the center of the device, the user can easily access the storage racks 16a-16d on both sides of it.

Hence, and as mentioned above, the incubator advantageously has a vertical plane 48 that divides it into a first section 50a (left-hand section from the user's perspective) and a second section 50b (right-hand section). Vertical plane 48 extends perpendicular to user door 42 and through access door 44. It also extends through guide rail 38. In the shown embodiment, it also extends through vertical axis 36.

Advantageously, the storage racks 16a-16d and/or the shaker units 20a, 20b are arranged symmetrically on opposite sides of vertical plane 48.

A first subset of the storage racks, namely the storage racks 16a and 16c of the present embodiment, is located in first section 50a, and a second subset of the storage racks, namely storage racks 16b and 16d of the present embodiment, is located in the second section 50b.

Similarly, a first subset (in the present embodiment shaker unit 20a) of the shaker units 20a, 20b is located in first section 50a, and a second subset (in the present embodiment shaker unit 20b) of the shaker units 20a, 20b is located in second section 50b.

Vertical guide rail 28 with its support tower 30 is located between carriage 32 and user door 42, allowing robot unit 26 to access the storage racks 16a-16d in both sections 50a, 50b as well as the opening of access door 44 in the back.

### Second Embodiment

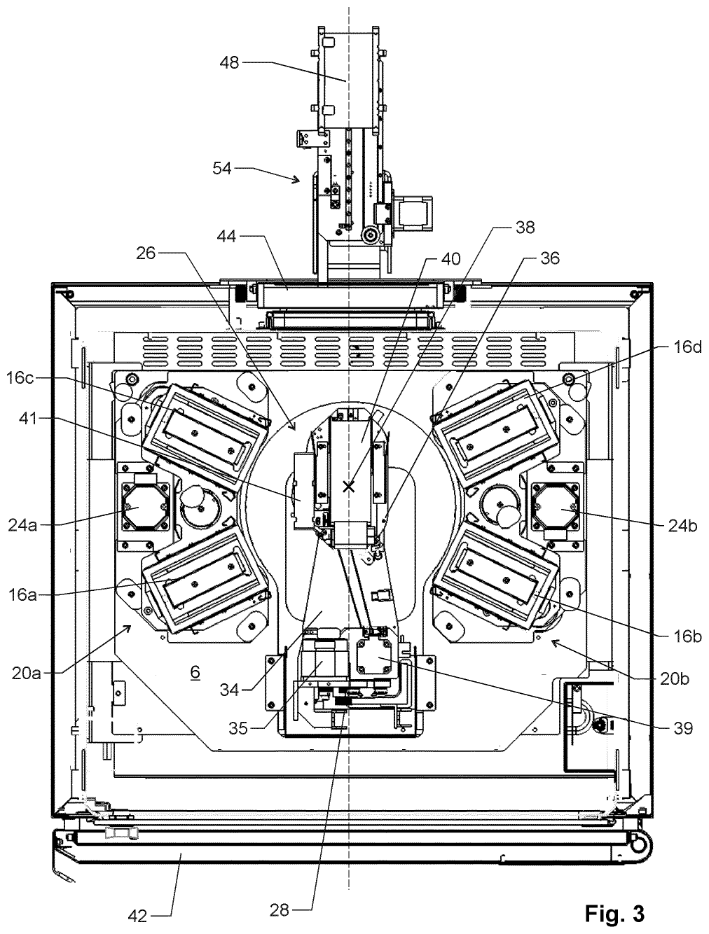

FIG. 3 shows a more detailed view of a second embodiment of the incubator from above, with the ceiling of chamber 6 removed.

The components of this embodiment basically correspond to the ones in FIGS. 1 and 2, and the design again is based on two shaker units 20a, 20b and four storage racks 16a-16d arranged around robot unit 26, again with a symmetric arrangement in respect to vertical plane 48.

FIG. 3 further shows a container station 54 located outside access door 44 for temporarily receiving sample containers as they are swapped between the incubator and an external system.

### Third Embodiment

FIG. 4 shows a further embodiment of an incubator, which comprises several shaker units 20a, 20b, 20a′, 20b′ and storage racks 16a-16d, 16a′-16d′ at different vertical levels 56a, 56b. This embodiment comprises two such levels 56a, 56b, but the design can be extended to three or more levels.

Guide rail 28 with its tower 30 vertically extends into both levels 56a, 56b such that carriage 32 can move to any of the storage locations 18 on any of the levels 56a, 56b.

### Method of Operation

As mentioned above, the shaker units 20a, 20b are operated to shake the storage racks 16a-16d and therefore the sample containers in the storage locations 18.

However, while robot unit 26 is to access a given storage location 18, control unit 14 stops the respective shaker unit 20a, 20b and resumes shaking once that access has ended. During this operation, the other shaker units can remain in motion.

### NOTES

In the embodiments above, shovel member (container access unit) 40 is rotatable about rotation axis 38. Alternatively or in addition thereto, robot unit 26 can be adapted to horizontally translate carriage 32 along a corridor extending between the storage racks 16a-16d, e.g. in a direction perpendicular to user door 42. This allows to increase the capacity of the incubator in a horizontal direction.

In the above example, each shaker unit carries two storage racks. However, a shaker unit may also carry more than two storage racks, such as three storage racks or even more storage racks. On the other hand, though, the number of storage racks per shaker unit should not be too large in order to customize the shaking to smaller subsets of racks. Advantageously, there may be between two and four storage racks per shaker unit.

Also, there may be more than two shaker units on a given level. If, for example, carriage 32 is translatable along a corridor as described above, there may be a row of two or more shaker units on each side of the corridor.

As mentioned, the vertical plane 48 divides chamber 6 into two sections 50a, 50b. The storage racks 16a-16d are advantageously arranged in either section but not in both, i.e. vertical plane 48 advantageously does not intersect any of the storage racks 16a-16d.

In some embodiments, the invention further comprises several shaker stations, i.e. several incubators, with each of them adapted to operate at different, individual operating parameters, such as at different amplitudes (throws) and/or frequencies (speeds).

While there are shown and described presently preferred embodiments of the invention, it is to be distinctly understood that the invention is not limited thereto but may be otherwise variously embodied and practiced within the scope of the following claims.

1. An incubator comprising:
a chamber,
a climate control unit connected to the chamber,
several storage racks arranged in the chamber,
wherein each storage rack comprises several storage locations arranged on top of each other,
a shaker unit comprising
a shaker drive and
a support member connected to the shaker drive and carrying at least two of the storage racks,
wherein the incubator comprises several separate shaker units.

2. The incubator of claim 1, further comprising
a user door providing access to the chamber, and
a robot unit arranged in the chamber, wherein the robot unit comprises
a) a vertical guide rail,
b) a carriage vertically displaceable along the guide rail,
c) an elevator drive connected to the carriage,
d) a container access unit movably arranged on the carriage,
e) an access drive connected to the access unit,
wherein a vertical plane extending perpendicularly to the user door, through the vertical guide rail, and through the carriage divides the chamber into first and a second section, with a first subset of the storage racks arranged in the first section and a second subset of the storage racks arranged in the second section.

3. The incubator of claim 2, wherein the vertical guide rail is located between the carriage and the user door.

4. The incubator of claim 2, further comprising an automated access door, wherein the vertical plane intersects the access door, and wherein the chamber is arranged between the access door and a user door.

5. The incubator of claim 4, wherein the robot unit is adapted to move sample containers between the storage locations and the access door.

6. The incubator of claim 2, wherein the first subset of the storage racks is mounted to a first subset of the shaker units and the second subset of the storage racks is mounted to a second subset of the shaker units.

7. The incubator of claim 2, wherein at any given vertical level, there is no more than one shaker unit in the first section and no more than one shaker unit in the second section.

8. The incubator of claim 1, further comprising several shaker units and storage racks at different vertical levels.

9. The incubator of claim 1, wherein the climate control is adapted to maintain a relative humidity of at least 60%.

10. A method for operating an incubator, wherein said incubator comprises:
a chamber,
a climate control unit connected to the chamber,
several storage racks arranged in the chamber,
wherein each storage rack comprises several storage locations arranged on top of each other, and
several separate shaker units, each shaker unit comprising:
a shaker drive, and
a support member connected to the shaker drive and carrying at least two of the storage racks,
the method comprising operating at least some of the shaker units at different speeds.

11. The method of claim 10, further comprising:
stopping the shaking of a first one of the shaker units while continuing the shaking of at least a second one of the shaker units, then
accessing one of the storage locations of the storage racks mounted on the first shaker unit with a robot unit, and then
resuming the shaking of the first one of the shaker units after ending the accessing.

12. The method of claim 10, further comprising maintaining a relative humidity of at least 60% in said chamber while operating the shaker units.

13. The incubator of claim 1, wherein the climate control is adapted to maintain a relative humidity of at least 90% in said chamber.

14. The method of claim 10, further comprising maintaining a relative humidity of at least 90% in said chamber while operating the shaker units.
